(19) United States
(12) Patent Application Publication
(10) Pub. No.: US 2019/0104967 A1
(43) Pub. Date: Apr. 11, 2019

(54) Anatomy Scanning System and Method

(71) Applicant: Tricia Dretzka-Kaye, Muskego, WI (US)

(72) Inventors: Tricia Dretzka-Kaye, Muskego, WI (US); Michael D. Marshall, Muskego, WI (US)

(73) Assignee: Tricia Dretzka-Kaye, Muskego, WI (US)

(21) Appl. No.: 15/743,141

(22) PCT Filed: Jul. 20, 2016
(86) PCT No.: PCT/US2016/043107

§ 371 Date: Jan. 9, 2018

Related U.S. Application Data
(60) Provisional application No. 62/196,402, filed on Jul. 24, 2015.

(51) Int. Cl. A61B 5/107; A61B 5/00; A61J 11/00

(52) U.S. Cl. CPC A61B 5/1077; A61B 5/4312; A61B 5/0062; A61J 11/005; A61B 5/6834

(57) ABSTRACT

A system and method for preparing a three dimensional model of anatomy such as a nipple using a scan of an actual nipple under vacuum pressure. The three dimensional digital model can be used to prepare a three dimensional physical model using three dimensional printing and other techniques.

### CROSS-REFERENCES TO RELATED APPLICATIONS

This application claims the benefit of prior-filed, co-pending U.S. Provisional Patent Application No. 62/196,402, filed Jul. 24, 2015, the entire contents of which are incorporated herein by reference.

### BACKGROUND OF THE INVENTION

The present invention relates to a scanning system and method and more particularly, to a system and method for scanning anatomy such as a mother's nipple for use in preparing a simulated nipple for use in connection with baby bottle nipples, pacifiers, and other implements associated with nursing.

Nursing a newborn baby, as well as babies of all stages, is the best practice and the most nutrient rich way to feed a baby. Mothers often have difficulty with babies latching on to their breasts right after birth. When such difficulty occurs, a nipple shield is often given to assist the baby with latching onto the breast. It is also common practice not to introduce any other style of nipple in the early stages of nursing to prevent nipple confusion. Nipple confusion is undesirable because it can cause great frustration for a mother and baby while nursing. The frustration of nipple confusion, coupled with the resulting sleep deprivation that a mother might experience, can cause a mother to cease nursing. Furthermore a mother may have to leave the home and/or return to work 6-12 weeks after the child's birth, which can cause fear and anxiety, especially if the baby has any difficulty with feeding from a bottle and or nipple confusion problems.

There exists a continuing need for creating a simulated nipple that closely mimics the actual anatomy of the mothers' nipple, which can offer benefits such as improving the process of latching on and preventing nipple confusion. If the bottle nipple mimics the anatomy of the mothers' nipple, the baby may be less likely to resist feeding from the bottle when compared with using a conventional nipple. This may make leaving the home an easier transition for mother and baby. In addition, the use of a nipple simulating the anatomy of the mother's nipple can enable easy alternations between actual breast feeding and bottle feeding.

### BRIEF SUMMARY OF THE INVENTION

The present invention provides a method and system for generating a simulated nipple that mimics the physical anatomy of a mother's nipple. The present invention further permits an efficient and cost effective way for producing such custom nipples. In one aspect, the present invention utilizes a vacuum chamber to scan the geometry of an erect nipple and produce an electronic model of the nipple. This electronic model can then be used to produce simulated nipples that mimic the geometry of the actual mother's nipple. Such custom simulated nipples can promote a variety of health and other benefits, including the promotion of breast feeding, prevention of nipple confusion, and other benefits. Other benefits of breast feeding include: more nutrient rich way to feed a child; decreased likelihood of illness; enhanced development of jaw, teeth, and facial structure; enhanced speech capabilities; better digestion when compared with formula; decreased risks of sudden infant death syndrome (“SIDS”) when compared with formula-fed babies. In addition, breast feeding has health benefits for the mother. Immediately after birth, the repeated suckling of the baby releases oxytocin from the mother's pituitary gland. This hormone not only signals the breasts to release milk to the baby (this is known as the milk ejection reflex, or “let-down”), but simultaneously produces contractions in the uterus. The resulting contractions prevent postpartum hemorrhage and promote uterine involution (the return to a nonpregnant state). In addition, there is a decreased risk of iron-deficiency anemia in the breastfeeding mother.

The system can easily be installed at a variety of convenient locations to enable a mother to have her breast scanned in a private, professional, and convenient environment and location. Such locations include, without limitation, medical clinics, physician's offices, pharmacies, retail outlets, and any other suitable location. The simulated nipples can be produced at a wide variety of locations, and there is no requirement that the scanning be conducted at the same location as the production facility. The digital model of the scanned nipple can be electronically transmitted to the manufacturing facility, and the simulated nipples can be shipped to the mother or any other desired location.

### BRIEF DESCRIPTION OF THE DRAWINGS

FIG. 1 is a perspective view of the system according to one aspect of the invention;

FIG. 2 is a side view of the system according to one aspect of the invention;

FIG. 3 is another side view of the system according to one aspect of the invention;

FIGS. 4A and 4B are front views of the system according to one aspect of the invention;

FIG. 5 is a perspective view of an optical emitted according to one aspect of the invention;

FIGS. 6A and 6B are front views of the system according to one aspect of the invention

FIG. 7 is a perspective view of a seal according to one aspect of the invention; and

FIGS. 8A, 8B, 8C, 8D, 8E, and 8F are illustrations of model preparation according to one aspect of the invention.

### DETAILED DESCRIPTION OF THE INVENTION

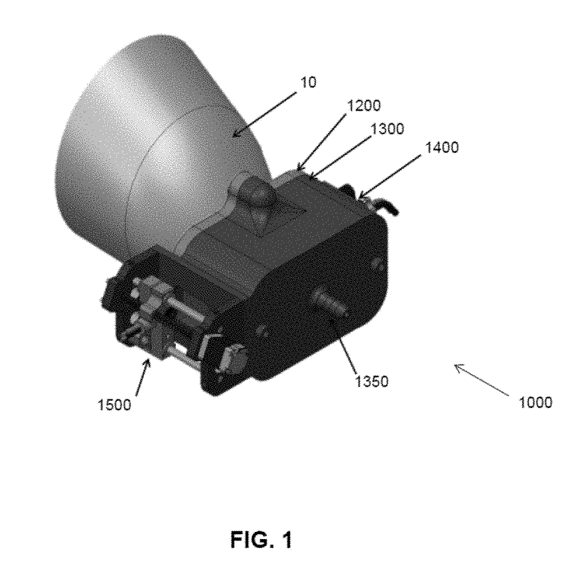

Referring to FIG. 1, a side view of a scanner system 1000 interfaced with a breast 10 is shown. Scanner system 1000 typically includes a seal 1200, a vacuum chamber 1300, an optical receiver 1400, and an optical emitter 1500. Seal 1200 is typically removably attached to chamber 1300. Vacuum chamber 1300 typically includes a vacuum port 1350 that can be interfaced with a vacuum or pump (not shown) or other implement that can be used to create a vacuum in chamber 1300. Optical receiver 1400 typically operates to record the contour of the nipple (not shown). Optical emitter 1500 typically emits the optical light (or other media) used to create the nipple contour recorded by receiver 1400. The light or other media used in connection with emitter 1400 and receiver 1500 can be selected from a variety of wavelengths including, without limitation, visible, infrared, and ultraviolet light.

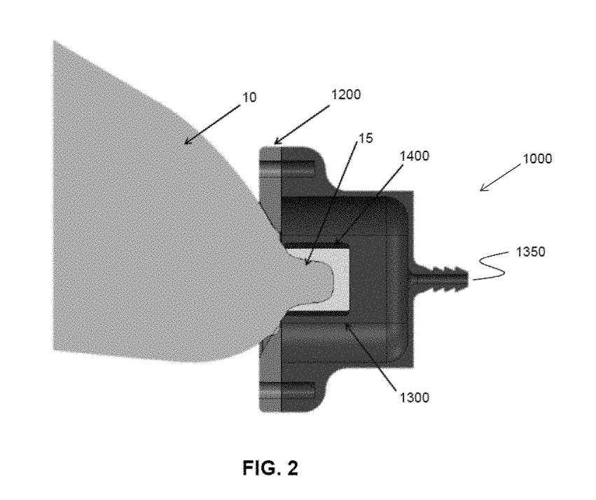

Referring to FIG. 2, a side view of system 1000 interfaced with breast 10 is shown. Breast 10 is typically positioned so that nipple 15 and a portion of breast 10 are inserted into optical chamber 1300. System 1000 is shown in cross-section, revealing optical receiver 1400. A vacuum is temporarily created in chamber 1300 by attaching a tube (not shown) to vacuum port 1350 and using a pump or other vacuum pressure means to remove the gas from the chamber 1300. In one aspect, atmospheric air is removed from chamber 1300. In other aspects, other gases such as nitrogen or another noble gas could be injected into, and then removed from chamber 1300. The resulting vacuum pressure created in chamber 1300 will cause nipple 15 to become erect, similar to the shape of the nipple when a child is nursing upon nipple 15. The shape of erect nipple 15 will be captured using optical receiver 1400.

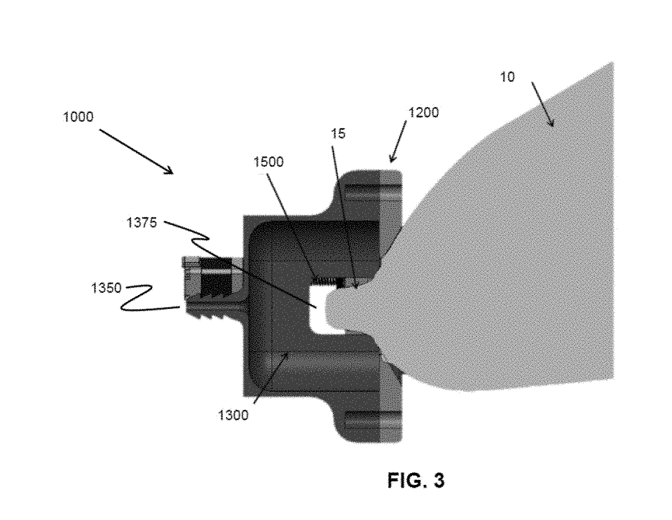

Referring to FIG. 3, another side view of system 1000 is shown. System 1000 is again shown in cross-section, revealing optical emitter 1500. The view of FIG. 3 shows the opposite cross section from that shown in FIG. 2. As with FIG. 2, a vacuum is temporarily created in chamber 1300 by attaching a tube (not shown) to vacuum port 1350 and using a pump or other vacuum pressure means to remove the gas from the chamber 1300. The vacuum pressure created in chamber 1300 will cause nipple 15 to become erect, similar to the shape of the nipple when a child is nursing upon nipple 15. Optical emitter 1500 will then scan the cross section of erect nipple 15. Optical emitter 1500 scans through transparent panel 1375. A wide variety of optical emitter and receivers can be used for receiver 1400 and scanner 1500. The receiver 1400 and scanner 1500 shown are for illustration only and are not intended to limited the devices that can be used in accordance with the present system.

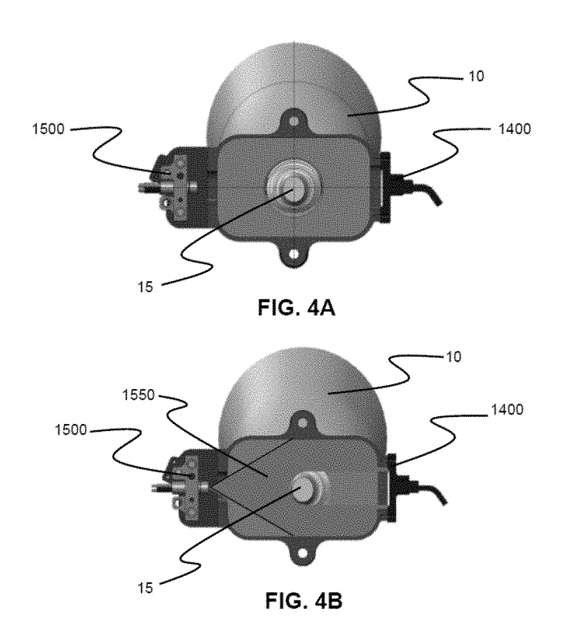

Referring now to FIGS. 4A and 4B, a frontal, cross-sectional view of system 1000 is shown. As shown, according to one aspect of the invention, a line laser 1550 projects from emitter 1500 and traces the outline, or contour, of erect nipple 15. The contour is recorded by optical receiver 1400. In another aspect, a macro-resolution camera could be used as receiver 1400, and either a line laser or bright light could be used as emission 1550. In another aspect, if the line laser 1550 becomes distorted (and cannot be compensated in software reconstruction), an optical diffuser (not shown) could be used to redirect the optical light or laser beam to the optical receiver. This optical diffuser (not shown) could be included as a separate component, integrated with panel 1375, or replace panel 1375

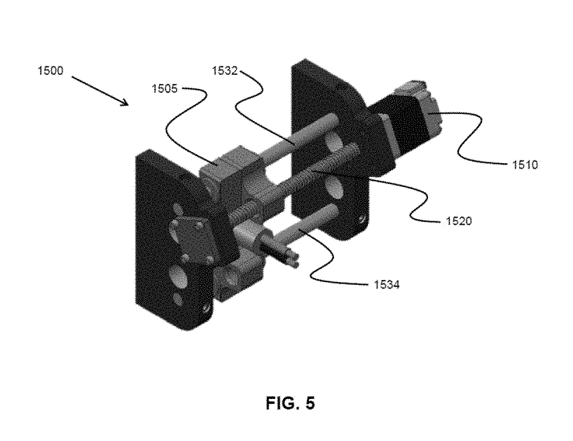

Referring now to FIG. 5, optical emitter 1500 according to one aspect of the invention is shown. The emitter shown in FIG. 5 is an assembly that can move along the longitudinal axis of the nipple (not shown). Emitter 1500 as shown in FIG. 5 includes a motor 1510 that rotates axle 1520, causing carriage 1505 to move along guiderails 1532 and 1534. In one aspect, motor 1510 is a stepper motor. Any other suitable motor could likewise be used for motor 1510, including a servo motor, hybrid motor, or linear screw motor. In one aspect, the position of carriage 1505 would be correlated to the signal picked up by receiver 1400, providing the contour of nipple 15 (not shown). In another aspect, vacuum pressure would be applied to chamber 1300 for the entire duration of a scan, and in the embodiment shown such duration would last for at least the amount of time it takes carriage 1505 to travel the longitudinal axis of nipple 15. In this aspect of the invention, it is assumed that the shape of nipple 15 is symmetrical about its longitudinal axis, which typically produced a shape sufficient for use in accordance with the present system.

In another aspect of the invention, a rotary device (not shown) could be used to take scans of the nipple at desired angles around the nipple axis. In this aspect, multiple scans would be required, and more complicated meshing software would be used to merge the multiple scans into a single, non-symmetrical nipple model. Such scanning would produce a simulated nipple that more closely resembles the actual geometry of nipple 15 when compared with the simulated revolved nipple produced using the system shown in FIG. 5.

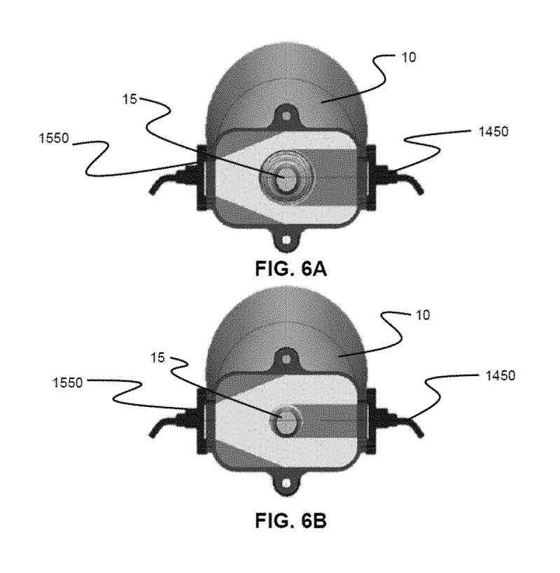

Referring to FIGS. 6A and 6B, an alternate optical emitter and scanner configuration according to another aspect of the invention is shown. Here, an optical emitter 1550 produces a uniform backlighting. Optical receiver 1450 receives the emitted light as either a picture, light-intensity matrix, or other similar optical pattern that shows the contrast between the bright backlighting and the obstruction (analogous to a shadow) created by the erect nipple 15 obstructing the backlighting. FIG. 6A shows the contrast created at the base of nipple 15 (i.e., where nipple 15 contacts breast 10), and FIG. 6B shows the contrast created towards the outer end of nipple 15.

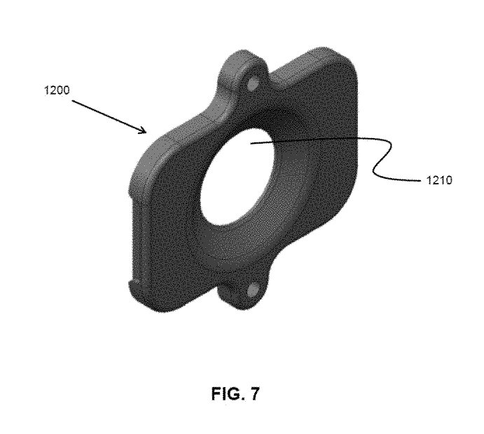

Referring now to FIG. 7, seal 1200 is shown. In one aspect, seal 1200 functions as a seal to allow vacuum pressure to be sustained inside chamber 1300. Seal 1200 is typically made from a flexible, medical, and/or food grade material. In one aspect, seal 1200 can be a urethane material with a SHORE A Durometer of 50 to 70. In other aspects, any other material suitable for creating a seal between breast 10 and chamber 1300 can optionally be used. Seal 1200 can optionally be either reusable or disposable depending upon a variety of considerations including, without limitation, cost, safety, regulations, hygiene, and aesthetics. In use, nipple 15 (not shown) is typically inserted through aperture 1210. Aperture 1210 is typically concave in shape to contact the outer surface of breast 10 (not shown) and form a seal. The diameter of aperture 1210 can optionally be sized to accommodate nipples 15 of differing diameters, which can range from approximately 21 mm to 36 mm. In one aspect, multiple seals 1200 can be designed for various aperture 1210 diameters. Such diameters can include 21 mm, 24 mm, 27 mm, 30 mm, and 36 mm. In addition, any other desired aperture 1210 diameters can be used.

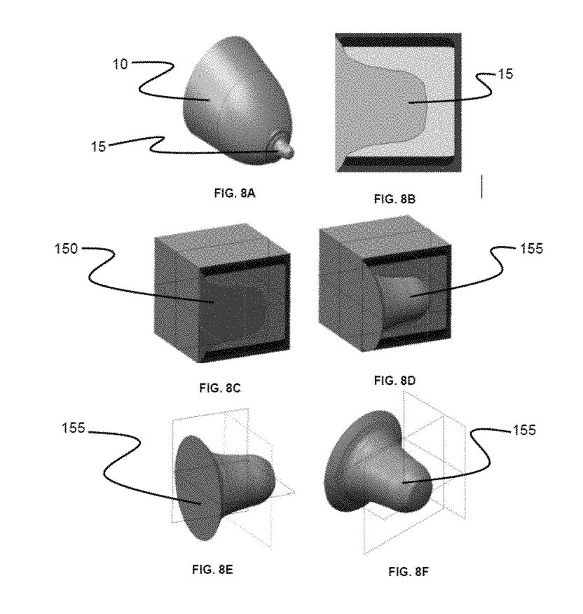

Referring now to FIGS. 8A, 8B, 8C, 8D, 8E and 8F, a non-limiting illustration according to one aspect of the present invention is shown for the various steps for creating the digital model of nipple 15. In step 1 (FIG. 8A), a mother's breast 10 and nipple 15 are shown. Step 2 (FIG. 8B) depicts the outline of nipple 15 prepared by optical recorder 1400 and emitter 1500 after nipple 15 has been inserted into chamber 1300 and vacuum pressure is applied to cause nipple 15 to become erect. In step 3 (FIG. 8C), a two-dimensional digital trace 150 of nipple 15 is prepared. In step 4 (FIG. 8D), this two-dimensional digital trace 150 is revolved around the longitudinal axis of trace 150 to prepare a three-dimensional digital model 155 of nipple 15. As shown in step 4 (FIG. 8D), digital model 155 is symmetrical about the longitudinal axis because the model is prepared from the two-dimensional trace 150. In step 5 (FIG. 8E), a side view of model 155 is shown. In step 6 (FIG. 8F), another rotated view of model 155 is shown.

In another aspect, the digital model 155 can then be used to produce a physical replication of nipple for use in connection with producing a bottle nipple, pacifier, or other device associated with nursing a baby. A wide variety of production methodologies can be used to produce the simulated nipple (not shown). Such methodologies include, without limitation, 3D printing, stereo lithography, and molding processes. A variety of materials can be used for the simulated nipples. Such materials include silicone and latex, and in certain aspects such silicone or such latex can be BPA free. Any other material suitable for a simulated nipple can likewise be used. In other aspects, materials suitable for three dimensional printing and having properties not inconsistent with the systems and methods described herein can likewise be used.

Following is one illustrative example of use of system 1000 to produce a simulated nipple based upon the anatomy of a mother's nipple. System 1000 is installed at physician's office, such as the office of a physician specializing in obstetrics and gynecology. A nursing mother can visit this office and have her breast and nipple scanned using system 1000. This procedure will typically take less than thirty minutes, and with proper scheduling and staffing can take less than fifteen minutes. The breast and nipple scan using system 1000 is conducted in a private, comfortable room that is clean and fully compliant with all rules and regulations governing medical clinics. System 1000 can generate a digital model 155 locally or transmit the collected scan data to another computer for remote processing.

The digital model 1000 can then be used to produce a series of simulated nipples using any one of the previously mentioned techniques. The simulated nipples could be produced on-site at the physician's offices, but more commonly the simulated nipples will be produced at another location and shipped to the mother. The mother can order as many simulated nipples as desired. In addition, the digital model, as well any other molds or other implements used to produce the simulated nipples, can be stored for producing additional simulated nipples at a later time. The nipples can be shipped to any desired location, including the mother's home or the physician's office.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

The description of the present technology has been presented for purposes of illustration and description, but is not intended to be exhaustive or limited to the invention in the form disclosed. Many modifications and variations will be apparent to those of ordinary skill in the art without departing from the scope and spirit of the invention. Exemplary embodiments were chosen and described in order to best explain the principles of the present technology and its practical application, and to enable others of ordinary skill in the art to understand the invention for various embodiments with various modifications as are suited to the particular use contemplated. It is understood that the invention is not confined to the particular construction and arrangement of parts herein illustrated and described, but embraces such modified forms thereof as come within the scope of the claims.

1-18. (canceled)

19. A system for scanning anatomical geometry, comprising:
a seal;
an aperture formed in the seal; and
a chamber formed by a structure,
wherein an optical emitter is disposed at a first position on the chamber,
an optical receiver is disposed at a second position on the chamber,
the seal is interconnected with the chamber,
a vacuum port is interconnected with the chamber, and
the chamber includes a plate disposed generally opposite the seal.

20. The system of claim 19 wherein the optical receiver is disposed at a position generally opposite the first position.

21. The system of claim 19 wherein the vacuum port is disposed on the plate.

22. The system of claim 19 wherein the plate is removable from the chamber.

23. The system of claim 19 wherein the plate is integral with the chamber.

24. The system of claim 23 wherein the vacuum port is formed in the plate.

25. The system of claim 19 wherein the optical emitter emits visible light.

26. The system of claim 19 wherein the optical emitter emits infrared light.

27. The system of claim 19 wherein the optical emitter moves along an axis while emitting a signal.

28. The system of claim 19 wherein the optical emitter remains stationary in relation to the chamber while emitting a signal.

29. The system of claim 19 wherein the aperture is concave in shape.

30. A method for scanning anatomical geometry, comprising:
inserting a nipple and breast portion through an aperture formed in a chamber formed by a structure;
creating a vacuum pressure in the chamber;
scanning at least portions of the three dimensional shape of the nipple while under vacuum pressure; and
creating a digital model of the nipple.

31. The method of claim 30 further comprising:
creating a three dimensional physical model of the nipple.

32. The method claim 31 wherein the three dimensional physical model is composed from silicone.

33. The method of claim 32 wherein the silicone is BPA free.

34. The method of claim 30 further comprising:
creating a three dimensional physical model of the nipple using a three dimensional printer.

35. The method of claim 30 wherein the aperture is adjustably sized to fit the diameter of the nipple and a portion of a breast.

36. The method of claim 35 wherein the aperture is adjustably sized by using interchangeable seal components having apertures with varying diameters.
